(19) United States
(12) Patent Application Publication
(10) Pub. No.: US 2013/0303842 A1
(43) Pub. Date: Nov. 14, 2013

(54) BLOOD VESSEL GRAFTS FOR REPAIR, RECONSTRUCTION AND FILLER MATERIALS

(71) Applicants: Steven M. Zeitels, Newton, MA (US); John C. Wain, Milton, MA (US)

(72) Inventors: Steven M. Zeitels, Newton, MA (US); John C. Wain, Milton, MA (US)

(73) Assignee: THE GENERAL HOSPITAL CORPORATION, Boston, MA (US)

(21) Appl. No.: 13/817,275

(22) PCT Filed: Sep. 2, 2011
(86) PCT No.: PCT/US11/50388

§ 371 Date: Jul. 29, 2013

Related U.S. Application Data
(60) Provisional application No. 61/379,838, filed on Sep. 3, 2010.

(51) Int. Cl. A61F 2/06

(52) U.S. Cl. CPC A61F 2/06
USPC 600/36

(57) ABSTRACT

Compositions comprising aortic material and methods for using harvested blood vessels (e.g., arteries or veins e.g., aortae) graft procedures, e.g., for restoring and repairing luminal structures (e.g., in the aerodigestive tract, such as the larynx, pharynx, and esophagus); as tissue fillers (e.g., injectable morselized blood vessel tissues, for use in cosmetic applications and functional sphincter enhancement); and as biological structural supports (e.g., intact portions of aortae, for use in repairing abdominal wounds, anastomoses, bony repairs, and the heart).

### CLAIM OF PRIORITY

This application claims the benefit of U.S. Provisional Patent Application Ser. No. 61/379,838, filed on Sep. 3, 2010, the entire contents of which are hereby incorporated by reference herein.

### TECHNICAL FIELD

The present invention relates to substances and methods for using a blood vessel (e.g., an artery, such as the aorta) as a material in graft procedures, including for reconstruction of luminal organs, and for use as a biological structural support, or for repairing or reinforcing a wound. Also described are the use of blood vessels (e.g., veins or arteries) to produce tissue fillers that have a number of uses, including to expand soft tissues.

### BACKGROUND

Sections of blood vessels, e.g., arteries such as the aorta, may be provided with other tissue structures that are used for certain transplants or reconstructive procedures, such as replacement of aortic valves. Some or all of such vascular material is often discarded or unused, with only certain portions of the tissue structures being used for a particular procedure.

It has been observed that circumferential tubular sections of aorta have been used with marginal success to replace circumferential tubular sections of the trachea or bronchial passages (see, e.g., Radu et al Ann Thorac Surg. 90(1):252-8, 2010). In humans, such procedures have required the use of permanent stents to maintain sufficient opening of the affected passageway (see, e.g., Wurtz et al, N Engl J Med. 355(18):1938-40, 2006). Aorta was presumably selected for these procedures primarily due to its similar tubular architecture to the trachea (see, e.g., French, Martinod et al C R Acad Sci III. 323(5):455-60, 2000). Tracheal replacement using an aortic graft is described, e.g., in Davidson et al., “Tracheal Replacement with an Aortic Homograft,” Ann. Thorac. Surg. 2009; 88:1006-8. It was observed in some animal models that tracheal implantation of such aortic material could, in some cases, lead to some tracheal epithelium growth on the aortic material. Development of some cartilage-like tissue in the aortic graft material has also been observed. Such effects are described, e.g., in Martinod et al., “Tracheal Regeneration Following Tracheal Replacement with an Allogenic Aorta,” Ann. Thorac. Surg. 2005; 79:942-9, which describes animal experiments in sheep. Although these observations suggested that aortic material can potentially be beneficially integrated with surrounding living tissue when implanted, prior tracheal reconstruction results in animal models were not reliably translatable to humans, and the synthetic tubular stents that were placed within the aortic graft lumen in human subjects could not be removed or the airway would collapse, as there was no vascularization of the grafts and a subsequent loss of mural stiffness related to the lack of vascularization.

### SUMMARY

The present invention is based, at least in part, on the discovery that blood-vessel derived grafts have a variety of uses in repairing, reconstructing, and reinforcing damaged tissues and organs. For example, processed blood vessels can be used as an injectable filler in living tissues, e.g., for cosmetic soft-tissue expansion or to enhance valvular and/or sphincteric function. In addition, as described herein, a blood vessel patch graft can be used to successfully repair defects in the larynx and pharyngo-esophagus. Furthermore, subjects who undergo this method generally recover airway, swallowing and phonatory function. From reconstructing this highly challenging scenario it is clear that blood vessel patches are an excellent organic biological substrate to support and/or reinforce a variety of wounds or repairs, in a spectrum of surgical scenarios in which inorganic synthetic materials are presently used. For example, these blood vessel grafts can be used to repair or support a repair of a number of organs such as the heart, uterus, stomach, bladder, diaphragm (e.g., in the case of a hiatal hernia), or chest wall. The blood vessel grafts described herein could also be used to bridge a wound dehiscence anywhere where there is soft tissue loss, such as the abdominal wall after infection. These blood vessel grafts can also be used as an onlay patch, e.g., to support the dura mater of the brain, or to prevent cerebrospinal fluid leakage after posterior skull-base otologic surgery, anterior skull-base paranasal sinus surgery, or generalized brain-related neurosurgery.

In a first aspect, the invention provides tissue filler compositions comprising particles of blood vessel in the range of 0.1 to 1000 micrometers. In some embodiments, there are substantially no (e.g., no more than 1% w/w) discrete pieces (particles) of blood vessel larger than 1000 micrometers, or no larger than 500 um, or no larger than 100 um, or no larger than 50 um. In some embodiments, there are substantially no (e.g., no more than 1% w/w) pieces smaller than 10 um, 1 um, 0.5 um, or 0.1 um. The pieces of blood vessel in the filler can thus fall within a range of sizes with the end points as above. In some embodiments, the pieces in the filler will be in the range of 0.1-1000 um, 0.1-100 um, or 1-50 um.

In another aspect, the invention features methods for preparing a tissue filler composition. The methods include providing a sample comprising a blood vessel or portion thereof; optionally freezing the sample; and processing the sample, to produce a tissue filler composition. Processing can include any method the reduces the sample to a form suitable for injection, e.g., a filler comprising particles of blood vessel as described herein. Methods for processing include morselizing, mincing, grinding, milling, pulverizing. Also provided are compositions prepared by the methods described herein.

In some embodiments, the compositions further include an aqueous solution or physiologically acceptable hydrogel.

In some embodiments, the compositions described herein are provided fully hydrated (e.g., in a ready to use injectable solution, e.g., a gel, paste, or liquid). In some embodiments, the compositions described herein are provided in lyophilized form.

In another aspect, the invention provides the use of the compositions described herein in a method for improving function of a valve or sphincter of an organ, the method comprising injecting the compositions adjacent to the valve or sphincter, to reduce the lumen of the valve or sphincter. In some embodiments, the valve or sphincter is a urethral sphincter, velopharyngeal sphincter, upper esophageal sphincter, lower esophageal sphincter, pyloric sphincter, ileocecal sphincter, anal sphincter, vocal cord glottic valve, aortic valve, mitral valve, or tricuspid valve.

In another aspect, the invention provides the use of the compositions described herein in a method for filling, raising, or supporting an area of soft tissue, the method including injecting the compositions into tissue under the soft tissue.

In some embodiments, the composition is injected intradermally, subcutaneously, intrafascially, intramuscularly, intramurally (e.g., sphincter injection), transmurally (e.g., hiatal hernia repair) and intravascularly (e.g., into heart valves). In some embodiments, the soft tissue is wrinkled, scarred, depressed, cheek, or lip skin. In some embodiments, the soft tissue is depressed due to a chronic disease, trauma, or surgical removal.

In an additional aspect, the invention provides methods for repairing, reconstructing, or reinforcing a damaged or weakened tissue or organ in a subject. The methods include providing a blood vessel patch sized to fit a damaged or weakened area in the tissue or organ; attaching the blood vessel patch to the area; and optionally attaching a vascularized soft tissue flap to the patch, thereby repairing, reconstructing, or reinforcing a damaged or weakened tissue or organ.

In some embodiments, providing the blood vessel patch comprises providing a blood vessel or portion thereof; and slicing the blood vessel or portion thereof longitudinally to provide a patch.

In some embodiments, the damaged or weakened tissue or organ is a luminal structure of the aerodigestive tract such as the laryngeal, pharyngeal or esophageal passages, and the methods do not include the use of a permanent stent.

In some embodiments, the damage is the result of trauma or a surgical procedure to remove a lesion, e.g., a malignant or benign tumor.

In some embodiments, the defect is due to a congenital malformation.

In a further aspect, the invention provides methods for repairing or reconstructing a stenotic region of a laryngeal, pharyngeal or esophageal, passage. The methods include incising a stenotic region of at least one of a larynx, a pharynx, or an esophagus; preparing a blood vessel patch sized to expand the stenotic region; and affixing at least one blood vessel patch to the incised region to increase a luminal caliber of the larynx, pharynx or esophagus. The methods do not include the use of a permanent stent to maintain the airway.

In some embodiments, the methods described herein include attaching a vascularized soft tissue flap to the patch. In some embodiments, at least a portion of the damaged or weakened area is vascularized soft tissue, and the patch is at least partially attached to the vascularized soft tissue.

In some embodiments, the methods include freezing and thawing the portion of aortic material prior to attaching the portion of material.

In some embodiments of the methods described herien, the blood vessel or portion thereof is an aorta or portion thereof

In some embodiments, the blood vessel or portion thereof is cryopreserved.

In some embodiments, the blood vessel patch is cryopreserved.

The blood vessel grafts and fillers described herein have several advantages. For example, there is a lack of tissue reactivity/antigenicity due to the use of autologous or allograft material or processing of the graft material prior to implantation. The grafts can be incorporated into recipient tissue at a variety of sites and in a wide range of contexts (e.g., even in tissues damaged by radiation or infection). The graft material persisted even during infection or cancer formation, maintaining its structural integrity. The graft material has the ability to act as a scaffold for epithelium and for epithelial appendages, e.g., nerves or mucus glands. The material has the ability to result in cartilage formation. Finally, the three dimensional geometry and/or consistency of the material can be varied to apply to a wide range of clinical circumstances.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1 is an exemplary image of an extensive glottic/subglottic cancer recurrence after failed radiotherapy treatment.

FIG. 2A is an exemplary image of a suspension microlaryngoscopy with laser incision of the left arytenoid and posterior glottic musculature.

FIG. 2B is an exemplary image of a suspension microlaryngoscopy with laser incision of the right arytenoid and posterior glottic musculature.

FIG. 3 is an exemplary image of a laryngotracheal defect after a tumor resection in which the anterior larynx and the anterior upper trachea were removed. A substantial laryngeal airway defect is visible in the image. The endotracheal tube is noted in the trachea at the inferior aspect of the image.

FIG. 4A is an exemplary image of an aortic conduit prior to reconfiguration as an aortic graft for use in reconstruction.

FIG. 4B is an exemplary image showing preparation of the aortic conduit by slicing it open longitudinally, to be used as an aortic graft “patch” for reconstruction as described herein.

FIG. 4C is an exemplary image of a prepared aortic graft from which the valve apparatus has been removed, which may be further cut to size and used for insertion into a laryngotracheal defect or other anatomical structure for repair or reconstruction, as described herein.

FIG. 5A is an exemplary image showing a reconstructive implantation of an aortic graft into a laryngotracheal defect. At this point, the aortic patch has been sutured to the caudal aspect of the airway defect.

FIG. 5B is an exemplary image of the aortic patch after being sutured in place to close the laryngeal airway defect, providing a complete reconstruction of the passageway with an aortic graft.

FIG. 5C is an image of the supraglottis and glottis subsequent to healing and epithelialization of the aortic graft.

FIG. 5D is an image of the subglottis subsequent to healing and epithelialization of the aortic graft.

FIG. 6A is a pharynx defect subsequent to total laryngectomy with a salivary bypass tube extending from the oropharynx to the esophagus

FIG. 6B is an exemplary image of an aortic conduit prior to reconfiguration as an aortic graft for use in the pharyngo-esophageal reconstruction, after being sliced open longitudinally to form a flat “patch” or “sheet” and before removal of the valve apparatus.

FIG. 6C is an exemplary image showing a reconstructive implantation of an aortic graft into a pharyngo-esophageal defect. At this point, the aortic patch has been sutured to the left lateral aspect of the pharyngo-esophageal defect.

FIG. 6D is an exemplary image of the aortic patch after being sutured in place to close the pharyngo-esophageal defect, providing a complete reconstruction of the region with an aortic graft.

FIG. 7A is a photograph of homogenized aorta paste in a syringe prior to injection.

FIG. 7B is a photograph of the appearance of the aorta paste 21 days after subcutaneous injection 21. Note the absence of tissue reaction as visualized through the transparent fascia and the similar color to injection material.

FIG. 7C is a photomicrograph of aorta paste injected superficial to the thyroarytenoid muscle of the larynx in a day 0 animal The injected aorta formed a well-circumscribed mass of eosinophilic fibrous material in which many small pieces of aorta can be observed. Little or no nuclear material was observed in the graft material at day 0, which is consistent with the decellularizing process by which the grafts are prepared.

FIG. 7D is a photomicrograph of aorta paste injected subcutaneously just beneath a thin muscle layer.

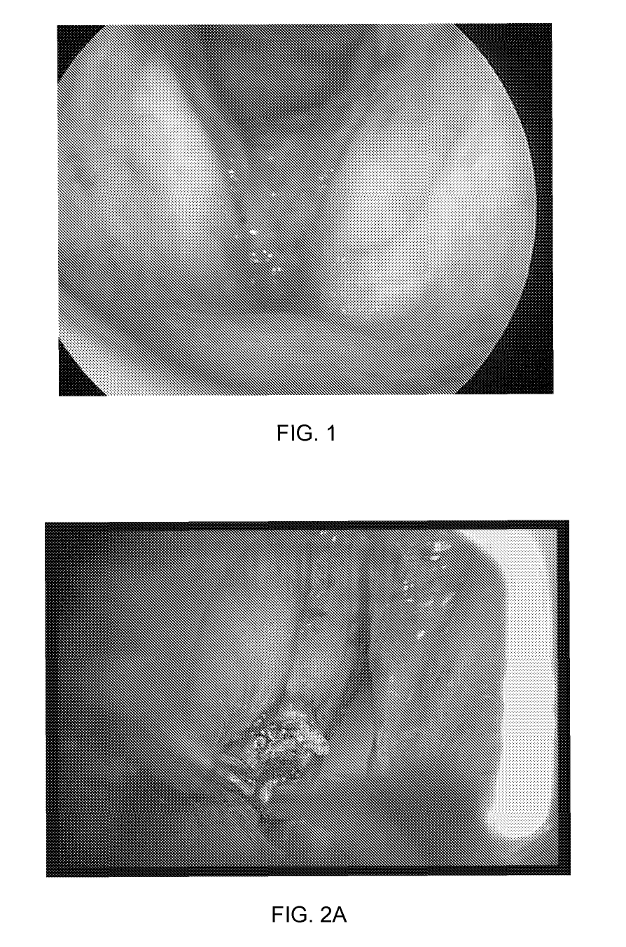

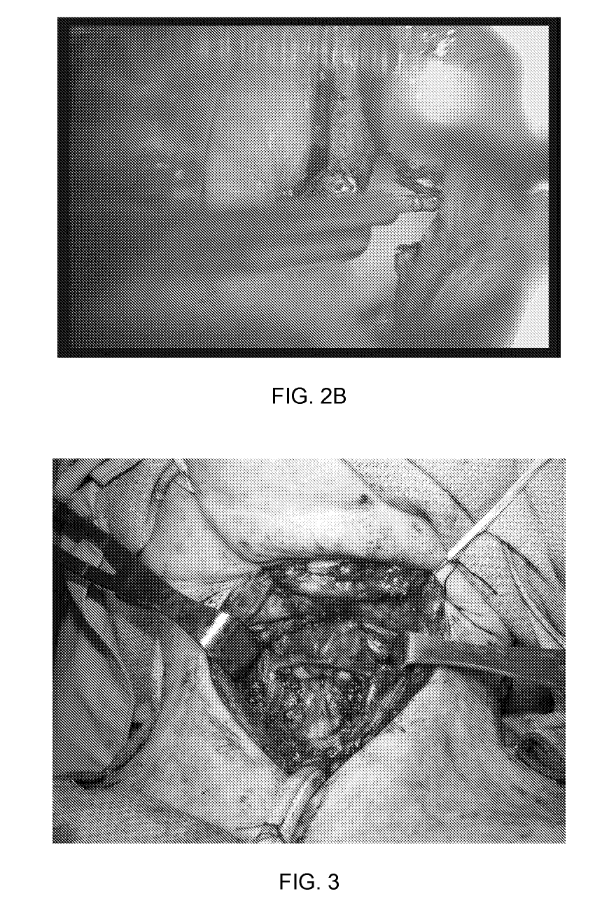

### DETAILED DESCRIPTION

As described herein, blood vessel walls are made of highly resilient and elastic material that incorporates easily with living tissue. Flat sheets, prepared by slicing the blood vessels longitudinally, integrate readily into a graft site if they are attached or adherent to suitable surrounding soft tissue which supplies angiogenic ingrowth to the graft, such as vascularized muscle, fascia, omentum or granulation tissue. The vessels can also be morselized and used as an injectable filler as described herein.

Materials derived from aorta have a variety of medical uses, including in the repair or reinforcement of wounds or other defects, to repair luminal defects in laryngeal, pharyngeal, and esophageal tissues, and as tissue fillers (e.g., injectable formulations).

### I. Preparation of Blood Vessel Grafts

The blood vessel grafts used in the present methods are preferably prepared from arteries, but in some embodiments are from veins. In some embodiments, e.g., where more structure or strength is desired, the grafts are prepared from arteries, e.g., aortae or other large arteries. In some embodiments, e.g., where a softer graft is desired, the grafts are prepared from veins, e.g., vena cava, iliac vein, renal vein, or pulmonary vein.

The grafts can be prepared from any source, e.g., human or non-human animals. In some embodiments, the grafts are homografts (also known as allografts) prepared from vessels harvested from human cadavers. In some embodiments, the grafts are xenografts prepared from vessels aortae, harvested from non-human, e.g., pig, bovine, horse, goat, or other mammal, cadavers. Particularly in embodiments in which the grafts are from non-human animals, the grafts are treated to render them non-antigenic (e.g., by fixation or decellularization), or are from genetically engineered pigs. In some embodiments, the grafts are autologous, i.e., harvested from the subject to whom the graft will be administered, e.g., from a peripheral vessel that can be safely resected, e.g., the subclavian or internal iliac artery.

The vessels for use in methods described herein can be provided fresh, or can be provided frozen, e.g., cryopreserved, e.g., with the use of DMSO and/or glycerol, using methods known in the art such as those described in U.S. Pat. No. 5,336,616; Arnaud et al., J. Surg. Res. 2000; 89(2): 147-154; Kreitmann et al., Eur. J. Cardio-Thoracic Surg. 1997; 11(5) 943-952; Lang et al., J Thorac Cardiovasc Surg 1994; 108:63-67; Langerak et al., Transplant Int. 2001; 14(4) 248-255, all of which are incorporated herein by reference in their entirety.

The vessels can be decellularized, e.g., using methods known in the art such as those described in U.S. Pat. No. 5,336,616; US2010285587; US2003228692, all of which are incorporated herein by reference in their entirety. In preferred embodiments, the vessels completely lack intact living cells, and are non-antigenic (i.e., will not trigger an immune response or rejection response in the recipient).

The vessels can be sterilized and treated or maintained, e.g., in an antibiotic solution (e.g., antibiotic preservation) to reduce bioburden e.g., to remove bacteria, e.g., using methods known in the art such as those described in WO 2010105021; Kreitmann et al., Eur. J. Cardio-Thoracic Surg. 1997; 11(5) 943-952; Lang et al., J Thorac Cardiovasc Surg 1994; 108:63-67, all of which are incorporated herein by reference in their entirety. The grafts can be processed aseptically.

Additional methods for preparing a vessel for use in the present methods are described in U.S. Pat. Nos. 4,890,457; 4,597,266; 5,071,741; 5,110,722; 5,122,110; 5,613,982; 5,149,621; 5,145,769; 5,160,313; 5,171,660; 5,424,209; 5,632,778; 6,372,229; 5,899,936; 866686; 595571; 7,318,998; and 7,763,081, all of which are incorporated herein by reference in their entirety.

The starting material for the grafts (e.g., intact preserved vessels) can also be obtained commercially, e.g., from Cryolife (Kennesaw, Ga.), Lifelink Foundation (Tampa, Fla.), or the Musculoskeletal Transplant Foundation (Edison, N.J.).

Preparation of Blood Vessel “Patches”

In some embodiments, the methods include longitudinally opening the tube of the blood vessel and thereby using it as a sheet, referred to herein as a “patch,” e.g., for a luminal structure or other organ, and optionally surgically affixing vascularized soft tissue to the patch, e.g., to the outside wall of the patch.

The malleability of the blood vessel wall patch allows for reshaping, while maintaining optimal structural rheology to function as a framework substrate to reconstruct, repair, or reinforce an organ, including an organ with complex geometry, such as the human larynx. In addition, the patches can be sewn together to form larger sheets, e.g., to repair, reconstruct, or reinforce larger defects or weaknesses in tissues, or to act as an organ “sling”, e.g., to hold an organ or graft in place.

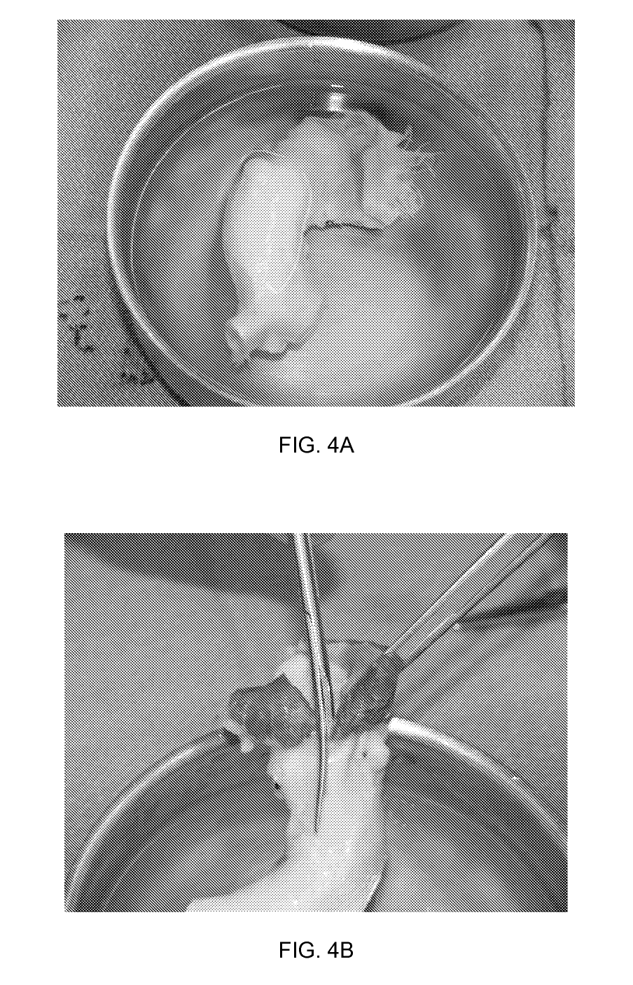

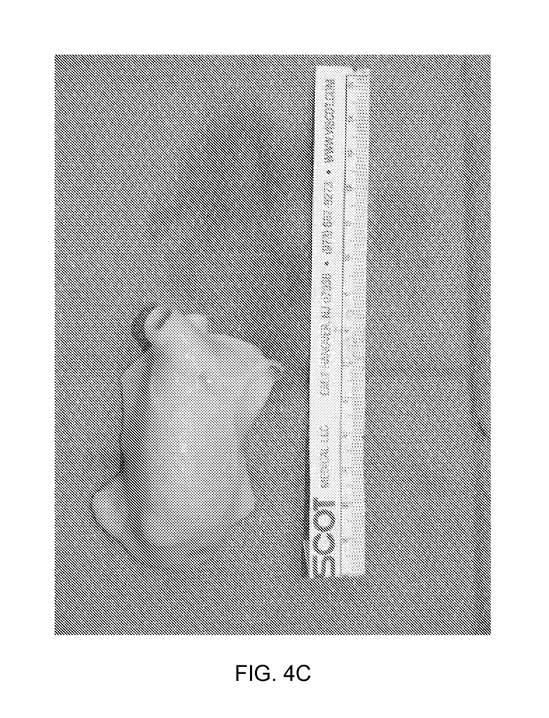

In some embodiments, the patches used herein do not retain the original tube shape, but rather have been “flattened” (though they are generally not, and need not be, absolutely flat) by slicing the vessel longitudinally (e.g., as shown in FIG. 4B). In some embodiments, after slitting the tube of the vessel longitudinally any attached valvular apparatus is removed as well.

In some embodiments, the patches are made using arteries, since they provide more structure than veins, and thus more tensile strength, e.g., for reinforcing weak tissues, or to support a mobile region or contracting muscular organ. In some embodiments, vein walls are used, e.g., after being meshed for coverage, where a softer, more mobile patch is desired.

In some embodiments, blood vessel walls in their native shape as a tubular structure (such as arterial, e.g., aortic, grafts) are used to repair defects (holes) or narrowing (stenosis) of anatomic luminal structures of the upper aerodigestive tract such as the larynx, pharynx, and esophagus. In some embodiments, the graft material may be treated with a drug or pharmacological agent to enhance healing and or incorporation of the graft into the recipient site, e.g., pro-angiogenic factors such as endothelial growth factors, as well as other trophic factors, such as nerve growth factor to encourage regional innervation of the patch or epithelial growth factors to accelerate re-epithelialization.

In some embodiments, the graft material may be mechanically altered to enhance healing and or incorporation of the graft into the recipient site in one or more ways, such as to by perforating it to allow for accelerated angiogenic in-growth or texturing the luminal surface for accelerated epithelial in-growth. Alternatively the patch may be meshed to increase the surface area that it may be used. Similar to using dermis, this could have applicability in covering larger wound surfaces such as after burns. Where tensile strength is not required, a blood vessel patch as described herein can be meshed to provide coverage of a greater surface area at the recipient site, with expansion ratios generally ranging from 1:1 to 6:1. The blood vessel patches can be meshed manually, e.g., using a scalpel, or using skin graft meshers, e.g., as described in Vandeput J, Nelissen M, Tanner J C, et al. 1995; 21(5):364-370; Taghizadeh R, Gilbert P M. Burns. February 2008; 34(1):109-110. Current meshers typically use one of two methods to mesh the graft. The first method is to use a plate, or carrier, to carry the graft under circular notched blades, as does the Zimmer Skin Graft Mesher and Mesh Dermatome (Zimmer, Inc., Warsaw, Ind.). The second method uses two opposing rollers that meet and cut the graft similar to how scissor blades cut paper. The Brennen Skin Graft Mesher (Brennen Med, St Paul, Minn.) uses this method. Other meshers include the Aesculap Power Systems Skin Graft Mesher (Aesculap Inc., Center Valley, Pa.); and the Rosenberg Adjustable Skin Graft Mesher (Robbins Instruments, Inc., Chatham, N.J.).

Preparation of Injectable Filler

The methods described herein also include the preparation of grafts that are usable as injectable soft tissue fillers. The fillers are prepared by processing the blood vessels into an injectable form. This can be done using any method that produces minced, morselized, or pulverized blood vessels, in pieces small enough to push through a needle, e.g., a needle as small as 33 gauge, or as large as 14 gauge, on the Stubs scale. In some embodiments, the blood vessel is processed into a relatively homogenous smooth filler, e.g., in which there are substantially no (e.g., no more than 1% w/w) discrete pieces of blood vessel larger than 1000 micrometers, or no larger than 500 um, or no larger than 100 um, or no larger than 50 um. In some embodiments, there are substantially no (e.g., no more than 1% w/w) pieces smaller than 10 um, 1 um, 0.5 um, or 0.1 um. The pieces of blood vessel can thus fall within a range of sizes with the end points as above. In some embodiments, the pieces in the filler will be in the range of 0.1-1000 um, 0.1-100 um, or 1-50 um.

Any method known in the art can be used to produce the fillers. In one exemplary method, blood vessels (i.e., blood vessel walls) are chopped into small pieces, e.g., manually chopped, and forced through one or more needles, e.g., a series of consecutively smaller needles, to produce a filler as described herein.

In another exemplary method, the blood vessels are optionally frozen and/or dehydrated, then processed, e.g., morselized, minced, ground, milled, or pulverized, to produce a paste as described herein. One of skill in the art would readily recognize that the parameters of the processing can be altered to produce a filler with larger or smaller pieces. A number of commercial machines are available for processing the blood vessels.

In addition, the size of the pieces can be varied to alter the mechanical characteristics of the filler. For example, a filler with larger pieces may have a stiffer, more resilient feel when injected. A filler with smaller pieces may be softer when injected.

In addition, the starting material can be altered to produce fillers with varying degrees of stiffness, resilience, and softness. For example, a starting material composed of veins, or a mixture of veins and arteries that is primarily veins, can be used to produce a softer filler, while a starting material that is composed entirely or primarily of arteries can be used to produce a filler that is stiffer and more resilient.

Since they are being processed, any caliber vessel can be used from the head, neck, torso or extremeties, and material derived from multiple vessels can be pooled.

The filler can optionally be combined with a carrier, e.g., a liquid, e.g., an aqueous solution, preferably saline, or a gel, e.g., a hydrogel or other physiologically acceptable gel, to form a filler with varied viscosity, to improve the handling or injectability of the material. Physiologically acceptable carriers are known in the art.

In some embodiments, the filler is provided in a paste or gel form. In some embodiments, the filler is provided in free-dried, cryopreserved, or lyophilized form. In some embodiments, the filler comprises additional ingredients, e.g., antibiotics or anti-inflammatory agents. In some embodiments, the filler is provided as a powder. In some embodiments, the filler is provided in a kit, comprising the filler, e.g., in lyophilized or dried form, with a sterile carrier to rehydrate the filler. In some embodiments, the filler is provided in a single-use syringe.

Methods of formulating suitable compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous injection can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions. For example, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size and by the use of surfactants. Prevention of the action of microorganisms can be achieved by various antibacterial and antifungal agents, for example, parabens, chlorobutanol, phenol, ascorbic acid, thimerosal, and the like. In some cases, it may be desirable to include isotonic agents in the composition.

Tracheal Repair using Aortic Grafts

Previous reports (Pressman and Simon, Surg Gynecol Obstet, 1958. 106(1): p. 56-62 (1958); Pressman and Simon, Am Surg, 25:850-6 (1959) used aortic homografts to reconstruct long-segment defects of the tracheal airway in canines in the 1950s. These experiments were done with an indwelling luminal stent. A human tracheal reconstruction with a carotid arterial autograft in a child a decade ago is described in Dodge-Khatami et al., J Thorac Cardiovasc Surg, 123(4):826-8 (2002). Subsequently, a circumferential aortic autograft (Azorin et al., Eur J Cardiothorac Surg, 29(2):261-3 (2006)) and allograft (Wurtz et al., N Engl J Med, 355(18): 1938-40 (2006)) reconstruction of the trachea was reported. However, all of these procedures required an indwelling luminal stent to maintain airway patency, and the patient described in Azorin et al. died.

Methods of Repairing or Reconstructing Laryngeal, Pharyngeal, and Esophageal Tissues

Reconstructing the larynx and pharyngo-esophagus with a blood-vessel graft patch presents unique reconstructive challenges. Inevitable coughing creates substantial intraluminal airway barotrauma, which could tear or dehisce the graft. The area has is not sterile and is colonized with substantial bacterial flora. Reflux of stomach acid and bile to the reconstructive site is routine and exposes the graft to a severe caustic exposure. There is substantial motion of the reconstructed region during generalized coughing and swallowing. Finally, the soft tissue in the recipient region is often poorly vascularized due to prior radiotherapy. Despite these substantial disadvantages, the present inventors have experienced surprisingly great success in reconstructing the larynx and pharynx, with the outside perimeter of the patch/sheet graft being in contact with local vascularized soft tissue.

Open transcervical partial laryngectomy (TPL) techniques have evolved since they were initially introduced for benign infectious disease 150 years ago (Ehrmann, Histoire des Polyps du Larynx. 1850, Strasbourg). In 1869 Solis Cohen (The Medical Record, 1869. 4: p. 244-247) reported performing a laryngofissure and partial laryngectomy for a malignancy, which was likely the first cure of laryngeal cancer (Zeitels and Healey, New England Journal of Medicine, 349(9):882-92 (2003)). Twentieth-century surgical innovations extended the volume of laryngeal tissue that could be removed while preserving swallowing function and an airway caliber without requiring a permanent tracheotomy (Jackson and Jackson, in Cancer of the Larynx. W.B. Saunders: Philadelphia. p. 216-230 (1939); Alonso, Transactions of the American Academy of Ophthalmology & Otolaryngology, 51:633-642 (1947); Leroux-Robert, Annals of Otology, Rhinology and Laryngology, 65:137 (1956); Laccourreye et al., Analysis of 240 cases. Ann Otol Rhinol Laryngol, 96(2 Pt 1):217-21 (1987)). In recent years, open TPL procedures have been performed less frequently due to a number of factors including increased use of endoscopic resection techniques using lasers (Jako, Laryngoscope, 82:2204-2215 (1972); Strong, Laryngoscope, 85:1286-1289 (1975); Vaughan et al., American Journal of Surgery, 136: 490-493 (1978)) and robotics (Weinstein et al., Ann Otol Rhinol Laryngol, 116(1):19-23 (2007)), as well as the popularity of chemotherapy-radiotherapy treatment regimens (Wolf et al., New England Journal of Medicine, 324:1685-1690 (1991)). Also, there is limited enthusiasm for TPL after failed radiotherapy because of wound-healing problems and impaired restoration of deglutition. Finally, there has not been consensus regarding surgical strategies for reconstruction of wide-field TPL defects that can reliably allow for tracheotomy decannulation. The present methods overcome these difficulties by using graft blood vessel (e.g. aorta) material, e.g., cadaveric cryopreserved homograft aorta, to reconstruct large laryngeal and pharyngo-esophageal defects, e.g., subsequent to oncologic resections and to increase the luminal caliber of the larynx, pharynx and esophagus when there is stenosis that precludes normal airway and swallowing function. Although, as described herein, there have been some preliminary reports over the last 50 years of the use of homograft blood vessels for tracheal repair, these results have been suboptimal and seldom done. There have been not been reports of using homograft blood vessels to repair the larynx, pharynx or esophagus.

Described herein are methods for the use of aortic grafts, e.g., fresh or preserved, e.g., cryopreserved, aortic grafts, e.g., xenografts or homografts, to reconstruct large-caliber laryngeal, pharyngeal, and esophageal defects, e.g., subsequent to cancer resections or for stenosis. Embodiments of the present invention can provide a method and materials for performing repair or reconstruction of anatomical tissues and structures, such as repairing portions of the larynx, pharynx, or esophagus alone or in combination, which may extend to include portions of the upper trachea. For example, the inventors have shown that aortic graft material can be effectively used to repair and reconstruct portions of the larynx and pharynx with successful results.

Reconstructive procedures were performed on 22 subjects to develop and establish the effectiveness of using aortic homograft material for laryngeal, pharyngeal and esophageal reconstruction. Sixteen of the 22 subjects required laryngeal cancer-resection reconstruction, three needed repair of laryngo-tracheal stenosis, and two required repair of pharyngo-esophogeal stenosis.

Twelve of the 16 cancer patients would have been appropriate candidates for total laryngectomy. All 12 had single-stage combined resection (transoral/transcervical) and reconstruction. They all were decannulated restoring an adequate laryngo-tracheal airway thereby not requiring a tracheotomy (artificial airway) and all recovered swallowing function (deglutition). Furthermore, all had a serviceable laryngeal sound source. A majority of these cancer patients with extensive tumors were successfully reconstructed despite presenting after having failed prior radiation treatment.

Three of the 22 patients underwent successful reconstruction of subglottic laryngeal stenosis, having achieved an adequate airway lumen without substantial vocal deterioration. Three of the 22 patients underwent successful reconstruction of pharyngo-esophagus to treat stenosis or to repair a cancer defect.

The aortic material used in these homografts was observed to retain an acellular scaffold and tensile strength. It also appears to be an inert material when used in grafting applications, exhibiting non-antigenic properties that need no additional immunosuppression treatment.

Thus, blood vessel (e.g., arterial, e.g., aortic) grafts as described herein can be used to repair defects resulting from open transcervical partial laryngectomy and/or pharyngectomy to remove benign or malignant tumors or lesions; repair of laryngotracheal stenosis; total laryngectomy for malignant tumors; and pharyngo-esophageal stenosis from cancer treatment (surgery and/or radiation), caustic exposure, inflammatory or infectious disease. As another example, blood vessel grafts can be used to repair defects resulting from trauma, e.g., penetrating trauma, to laryngeal tissues. As a further example, aortic grafts can be used to repair congenital laryngeal, pharyngeal and esophageal deformities, e.g., laryngeal and laryngotracheoesophageal clefts or fistulae. The methods for laryngeal, pharyngeal and esophageal repair described herein do not include the use of a permanent indwelling stent.

Methods of Repairing, Reconstructing, or Reinforcing

In further embodiments, portions of the blood vessel (e.g., arterial, e.g., aortic) wall can be used for various structural repairs, such as repairing, supporting (reinforcing) or reconstructing portions of the abdominal wall (e.g., for hernia repair procedures), the diaphragm (e.g., in the case of a hiatal hernia), the bladder, the stomach, the uterus, the chest wall or to repair the myocardium of the heart. The aortic homograft material has been observed to accept sutures well and is non-antigenic after being cryogenically treated. It can also provide a scaffold for growth of adjacent tissues to improve incorporation and continuity of cellular structures and systems while also providing mechanical strength, etc. The blood vessel grafts described herein could also be used to bridge a wound, e.g., a wound dehiscence, and anywhere where there is soft tissue loss or thinning, such as in the abdominal wall after infection.

For example, the blood vessel grafts can be used as an organic natural material in various reconstructive procedures, including many of those in which synthetic Gore-Tex® medical products are currently used. For example, blood vessel material can be used as a natural alternative for synthetic patches, e.g., to repair abdominal walls, for covering portions or entire surfaces of certain implants, supporting damaged or diseased eye sockets in reconstructive procedures (e.g., for fascia lata for the floor of the orbit). It can be used in neurosurgical procedure to support the dura mater of the brain and would be especially helpful in surgery, e.g., in brain surgery, e.g., of the anterior skull base (e.g., paranasal sinuses) and posterior skull base (e.g. temporal bone), to control cerebrospinal fluid leakage. Another application would be for coverage and protection of important structures of wounds in diabetics or after burns, radiation, immunosuppression, idiopathic inflammation or infection. Blood vessel grafts can also be used as a patch to reinforce an anastomosis as well as a repair of bone (cover hardware) or soft tissue (stomach or heart).

### II. Blood Vessel “Patches” for Repairing, Reconstructing, or Reinforcing Tissues and Organs

Although previous reports indicated that use of an intact aortic “tube” for tracheal repair was feasible in animals without the need for intraluminal stents, this success was not repeatable in humans (Wurtz et al J Thorac Cardiovasc Surg.

140(2):387-393.e2, 2010). The present observations reveal that those prior efforts did not recognize key procedural requirements for reliable success of blood vessel grafts to be incorporated into local tissues. In the previously reported attempts at mediastinal tracheo-bronchial aortic graft reconstruction, the graft environment lacked local soft tissues to supply angiogenic ingrowth into the graft. The absence of appropriate local soft tissues at the perimeter of the aortic trachoebronchial grafts compromised incorporation of these grafts and resulted in their lack of intrinsic structural integrity, as evidenced by the ongoing requirement for intraluminal stents to maintain patency (Wurtz et al J Thorac Cardiovasc Surg. 140(2):387-393.e2, 2010; Azorin et al., Eur J Cardiothorac Surg, 29(2):261-3 (2006)).

Furthermore, previous reports failed to recognize key constituent characteristics of graft blood vessels that allow them to be shaped, conformed and reprocessed for a large variety of surgical applications as described herein.

In contrast, the present methods, including methods of tissue and organ reconstruction and repair, produce successful results in humans. This made it clear that, despite previous evidence to the contrary, a cryopreserved nonantigenic blood vessel patch/sheet graft would be an excellent organic biological strategy to support and/or reinforce a variety of wounds or repairs in a spectrum of surgical scenarios in which inorganic synthetic materials are used, so long as a local source of soft vascularized tissue is present at the graft site. The local source can be immediately adjacent to or completely or partially surrounding the graft site, or can be close enough that a vascularized, e.g., pedicled, flap can be created and sutured to the vessel wall graft to provide angiogenic ingrowth into the graft. In some embodiments, the methods include transposition of one or more vascularized flaps into position to support the graft patch.

Thus, the methods include suturing a blood vessel graft as described herein into the desired location, and, if necessary, surgically creating a flap of vascularized soft tissue (e.g., a pedicled flap) and suturing the flap onto all or a portion of the graft.

### III. Methods of Using Injectable Formulations

Presently, there are limited organic injectable options for tissue fillers, for use in reconstructive or cosmetic use, or for enhancing valvular competence, e.g., vocal cords and/or sphincters such as in the bladder neck as well as the upper and lower esophagus. Dermis, collagen, and fat have various limitations in residence time in the recipient site, facility of delivery, reliability, and tunability of viscosity. The blood-vessel derived tissue fillers described herein provide an organic substrate as an injectable to fill tissue defects and or enhance valves and sphincters that could be naturally incorporated for longer residence time and potentially permanent repair.

As described herein, the tissue fillers integrated well when implanted in living tissue and are less likely to be resorbed over reasonable times than other common filler materials such as fatty tissue.

The fillers described herein can also be used for reconstructive and cosmetic purposes. In some embodiments, the fillers described herein are injected to smooth out fine lines on the surface of the skin, fill out deep lines (e.g., nasolabial folds), augment soft tissues (such as the lips), or even effectively augment facial bone structure (e.g., cheeks or chins). The fillers can be used to augment, or treat depressions or malformations in, soft tissues due to injury, trauma, congenital defects, infection, and surgery/oncologic resection (e.g., lumpectomy).

The fillers can be injected into any site appropriate to treat the condition, e.g., into or under the tissue to be repaired, e.g., at the desired transplant site, into or under dermis, muscle, or fascia, (e.g., intradermally, subcutaneously, intrafascially, intramuscularly, intramurally (e.g., sphincter injection), transmurally (e.g., hiatal hernia repair) and intravascularly (e.g., into heart valves).

The tissue fillers can also be used in other applications in which dissolution or absorption by the body is undesirable, such as enhancing sphincteric function of an organ. For example, the tissue fillers can be injected into one side of a vocal-cord glottic valve to facilitate phonation after vocal-cord paralysis (e.g., to narrow the distance between the cords), or if there is a tissue defect from trauma or a cancer resection. The tissue fillers may also be used to expand the soft palate, e.g., to reestablish velopharyngeal competency during speech and swallowing, which can be impaired congenitally or from neurological disorders as well as from tonsillectomy or the treatment of neoplasms. The tissue fillers could also be used, for example, as an injectable in the bladder to alleviate urinary incontinence (e.g., by injection into one side of the bladder neck to narrow the distance between the flaps of the bladder sphincter), in the esophagus at the upper or lower esophageal sphincter to control or reduce unwanted reflux. These examples are representative of sphincteric sites in the upper aerodigestive tract (UAT) and genitor-urinary systems (GUS) but morselized blood vessels may be used at any location within these anatomic regions (UAT, GUS) in which there is sphincteric function.

### EXAMPLES

The invention is further described in the following examples, which do not limit the scope of the invention described in the claims.

### Example 1

### Aortic Homograft Reconstruction of Partial Laryngectomy Defects

This example describes the use of cryopreserved aortic homograft to reconstruct large-caliber laryngeal defects subsequent to cancer resections

Materials and Methods

A retrospective case review was done on 16 patients who underwent large-volume transcervical partial-laryngectomy for cancer with single-stage aortic-homograft reconstruction of the defect. Institutional Review Board approval was obtained.

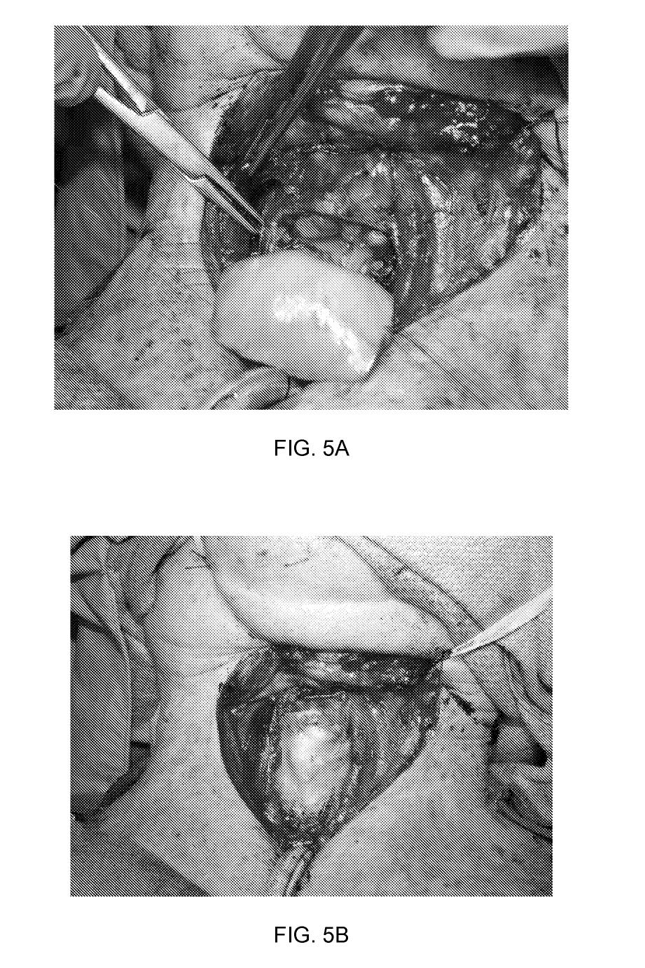

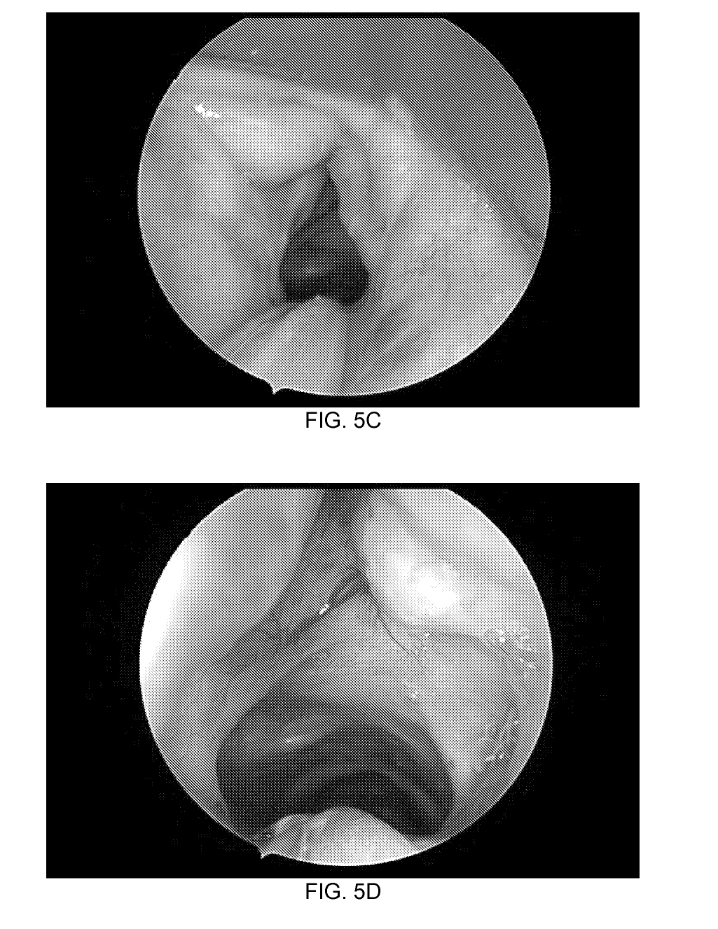

After standard resections were performed (FIG. 3) and clear frozen section margins were established, a commercially-available cadaveric cryopreserved aortic homograft (LifeNet Health, Virginia Beach, Va.) was affixed to the residual laryngo-tracheal cartilage. The aortic soft tissue has normal intrinsic curves (FIGS. 4A and 4C), which were utilized to ensure for intraluminal convexity to enhance the airway caliber. After excising the valve apparatus from the homograft, typically the ascending aortic segment was incised longitudinally (FIG. 4B) to create a convex onlay patch which was tailored to the surgical defect. (FIG. 4C) The graft was affixed extraluminally to extrinsic laryngeal strap muscles or rotated sternomastoid muscle, which provides for future angiogenic ingrowth and incorporation of the homograft into local soft tissues (FIGS. 5A-D). No patient received an intraluminal stent or pharmacologic treatment such as immunosuppression specific to managing the aortic soft tissue.

All patients had a tracheotomy placed intraoperatively to facilitate visualization of the cancer field and the resection. The tracheotomy tube cuff was left inflated for 8-12 days postoperatively to protect the graft anastomosis from disruption due to coughing in the early postoperative period.

Results

The resultant pathology revealed squamous cell carcinoma (12/16), chondrosarcoma (3/16), and synovial cell sarcoma (1/15). Nine of 16 had previously failed radiotherapy. At least 40% of the cricoid circumference was resected in 9/16 and one cricoarytenoid joint was removed in 7/16. All 16 patients had their tracheotomy tube decannulated and none developed postoperative subcutaneous emphysema. All 15 have laryngeal phonation while all resumed oral intake. One of 16 subsequently lost adequate deglutition subsequent to post-resection radiation.There were no major complications or wound infections related to the graft placement. Seven of 16 underwent endoscopic removal of granulation postoperatively.

Successful would healing was remarkable and uneventful considering that the aortic homograft is avascular and acellular. This is despite the fact that healing laryngeal cancer-resection defects poses unique challenges for optimal airway, swallowing, and phonatory function. The graft must remain intact without dissociating or necrosing to maintain the patency of the airway lumen, while only receiving slow angiogenic ingrowth from the muscles affixed to the graft's extraluminal surface. The structural integrity and position of the graft must also withstand intraluminal airway pressures of ˜120 cm of water during coughing (conversational voice requires ˜6 cm of water) once the tracheotomy tube is removed. Finally, the inside surface of the graft is exposed continuously to aerodigestive organisms as well as caustic exposure from likely laryngo-pharyngeal reflux that often penetrates into the laryngeal introitus.

No patient developed a major wound infection or fistula. All patients developed post-operative granulation on the intraluminal graft surface from 4-10 weeks after the reconstruction. It was not uncommon for this to be endoscopically removed in the operating room or the office with an angiolytic laser (Zeitels et al., Ann Otol Rhinol Laryngol, 115: 571-580 (2006); Zeitels et al., Ann Otol Rhinol Laryngol, 115:679-685 (2006)). This pattern of wound healing mirrors normal granulating wounds seen routinely after endoscopic resection of laryngeal cancer. Similar to this experience, it may take 3-4 months for the granulation to recede prior to epithelialization (Zeitels et al., Annals of Otology, Rhinology & Laryngology, 99: 951-956 (1990)) and typically longer if there has been prior radiotherapy. Areas of the graft that failed to adhere to extraluminal tissue were easily removed transorally during ablation of exuberant granulation. Other strategies may be used to accelerate the protracted period of wound healing evidenced by granulation prior to epithelialization.

All patients developed a lung-powered laryngeal phonatory sound source with a wide range of acoustic and aerodynamic characteristics. The voice outcome is not primarily related to the reconstructive technique but is more dependent on the laryngeal soft-tissue that could be oncologically preserved given the size and location of the cancer. No patient is using artificial phonation such as an electrolarynx. Using the aortic homograft to replace laryngeal cartilage framework scaffold restores and preserves key architectural structure of the larynx. The voice quality of all of these patients will likely be enhanced substantially over the next few years given the advanced development of subepithelial vocal biomaterials (Zeitels et al., New England Journal of Medicine, 349(9): 882-92 (2003); Zeitels et al., Ann Otol Rhinol Laryngol, 116 (Supplement 198): 1-16 (2007)).

### Example 2

### Pharyngo-Esophageal Reconstruction with a Vascular Aortic Homograft

Wound breakdown and fistula formation subsequent to total laryngectomy in radiation failure scenarios are commonplace. A variety of reconstructive strategies have been developed to avert this complication with varying degrees of success and complexity. Analogous wound healing issues were encountered during partial laryngectomy procedures, which were successfully remedied using cadaveric aortic homograft for large caliber cancer resection airway defects. The present example describes procedures to examine the value of homograft aorta for reconstructing the pharynx in an irradiated field.

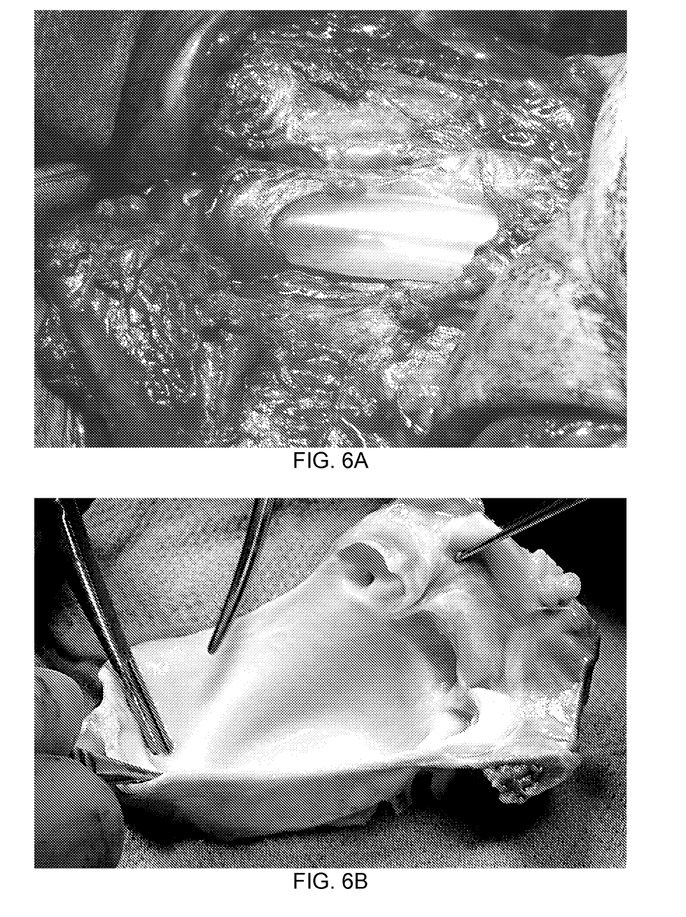

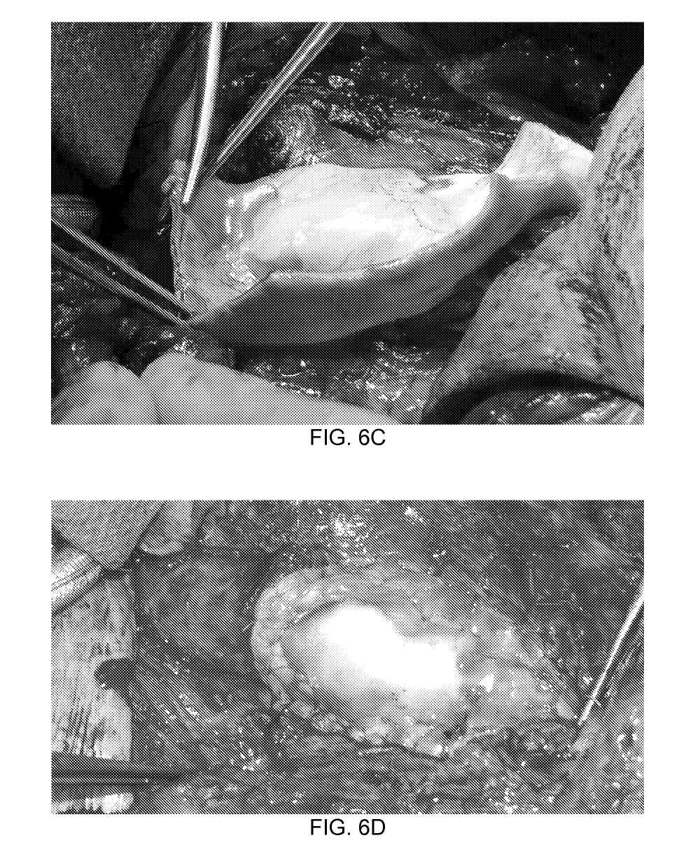

A total laryngectomy was performed in patients who had failed prior radiotherapy. A salivary bypass tube was positioned to traverse the oropharynx to the esophagus (FIG. 6A). Without using immunosuppression, a patch of homograft aorta prepared as described above was used to reconstruct the anterior wall of the neopharynx. Typically, after excising the valve apparatus from the homograft, the ascending aortic segment was used to create a convex onlay patch tailored to fit the surgical defect (FIG. 6B, 6C). The edges of the graft were sutured to the esophagus caudally the lateral pharyngeal walls inferior constrictor muscles bilaterally, and the tongue-base superiorly (FIG. 6D). Note the normal intrinsic curves of the aortic homograft, which were used to ensure the intraluminal convexity to enhance the pharyngeal caliber. Vascular supply to the homograft came from local muscle flap (e.g., infrahyoid extrinsic laryngeal muscles and/or sternocleidomastoid muscle) rotation. The salivary bypass tube was typically removed in approximately 3-4 weeks postoperatively. The aortic homograft provided a novel approach to reconstruction of the neopharynx in difficult irradiated regions, and also has potential for pharyngeal stenosis reconstruction.

In conclusion, effective reconstruction of the neopharynx with cadaveric homograft aorta after total laryngectomy was achieved in post radiation failure patients. The success of this approach is based on some or all of the following: ease of handling of the soft tissue graft substrate; lack of immunogenicity; and the practical incorporation of the aortic homograft into local soft tissues.

The encouraging results were similar to our broader experience in laryngotracheal airway reconstruction. The neopharyngeal reconstruction methods described are technically simple and within the skills of any head and neck surgeon.

### Example 3

### Injectable Aortic Filler

The use of aorta allograft material was tested as an injectable substance in a series of 8 guinea pigs.

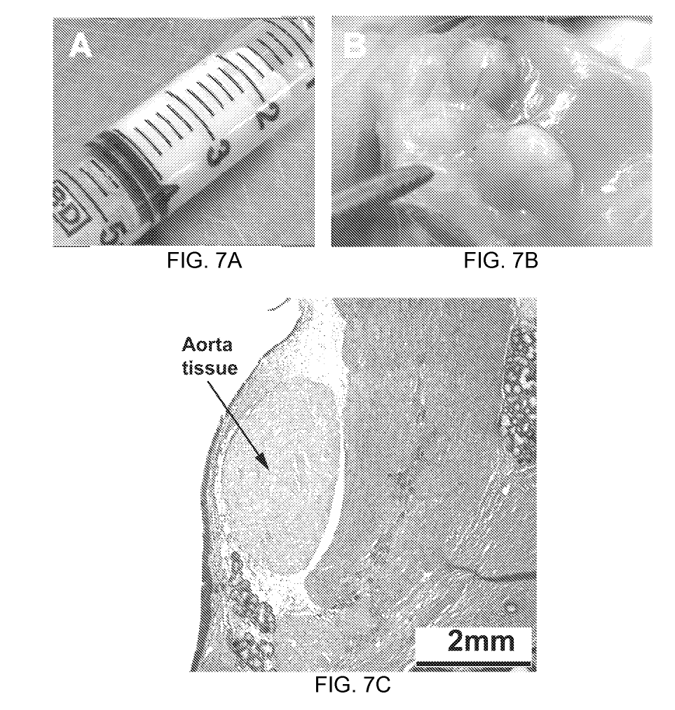

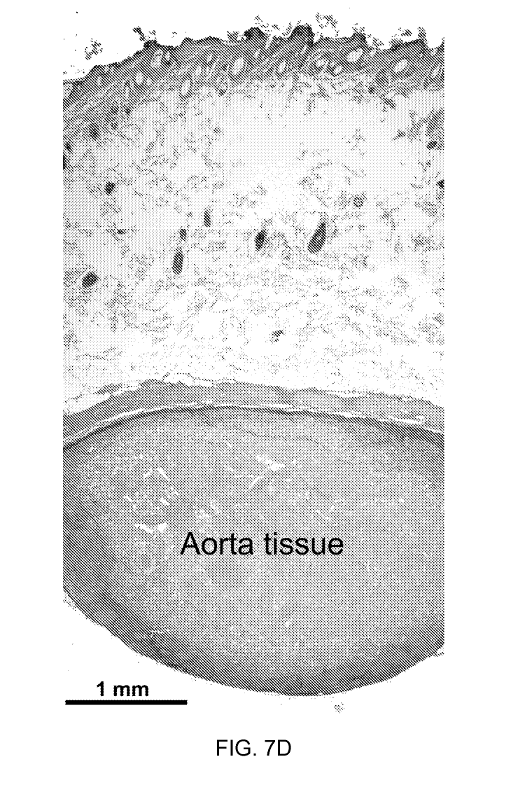

The aorta was converted to an injectable paste by cutting a sample of about 2 g into small pieces and then processing it in a cryogenic grinder (SPEX SamplePrep 6770 Freezer/Mill). The processed material was observed under a microscope to consist of small fibrous particles in the range of 1 to 50 micrometers and it had the consistency of a paste. It was loaded into syringes for injection (FIG. 7A) and used immediately or was kept in a −80° C. freezer until use.

Eight adult female guinea pigs were anesthetized with ketamine/xylazine and prepared for suspension microlaryngoscopy using a small custom-made speculum and a Leica operating microscope. The larynx was exposed and 12 microliters of the aorta material were injected into the pre-epiglottic space using a Hamilton syringe and 25 gauge spinal needle. Subcutaneous injections of 0.4 ml were also placed on the back of the animals using 23 gauge needles and 1 cc syringes. In some subcutaneous injections the aorta paste was combined 50/50 with Restylane or with a polyethylene glycol-based polymer. Animals were sacrificed at 0,1,3 and 5 weeks post-injection and the tissues were collected for histological processing and analysis.

In all cases it was observed that the aorta paste was homogeneous and easy to inject, either into the larynx or beneath the skin, through a small diameter needle.

After 7 or 21 days the appearance of the grafts was similar (FIG. 7B), except that at the latter time point, the periphery of the grafts was beginning to be invaded by host cells, including histiocytes and fibroblasts. There was no volume reduction and there was very limited inflammatory reaction at the host-graft interface.

FIG. 7D shows a section through subcutaneously injected aorta material after 21 days. The graft was injected just below the thin subcutaneous muscle layer (panniculus carnosus). A variety of sizes of injected chunks can be seen in the graft. White blood cells (WBCs) and fibroblasts have begun to invade the perimeter of the graft to depth of 100-300 micrometers, however, the gross architecture has not been substantially altered. In places they outline small chunks of the aorta tissue. There is a sharp boundary made up of closely spaced cells separating this ‘invasion zone’ and core of the graft. It looks like a line drawn with a pen in the figure. There are numerous small blood vessels (BV) that can be seen invading the graft.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A tissue filler composition, comprising particles of blood vessel in the range of 0.1 to 1000 micrometers.

2. A method of preparing a tissue filler composition, the method comprising:
providing a sample comprising a blood vessel or portion thereof;
optionally freezing the sample; and
processing the sample, to produce a tissue filler composition.

3. A composition prepared by the method of claim 2.

4. The composition of claim 1, further comprising an aqueous solution or physiologically acceptable hydrogel.

5. The composition of claim 1, which is lyophilized.

6. A method of improving function of a valve or sphincter of an organ, the method comprising injecting the composition of claim 1 adjacent to the valve or sphincter, to reduce the lumen of the valve or sphincter.

7. The method of claim 6, wherein the valve or sphincter is a urethral sphincter, velopharyngeal sphincter, upper esophageal sphincter, lower esophageal sphincter, pyloric sphincter, ileocecal sphincter, anal sphincter, vocal cord glottic valve, aortic valve, mitral valve, or tricuspid valve.

8. A method of filling, raising, or supporting an area of soft tissue, the method comprising injecting the composition of claim 1 into tissue under the soft tissue.

9. The method of claim 8, wherein the composition is injected intradermally, subcutaneously, intrafascially, intramuscularly, intramurally, transmurally and intravascularly.

10. The method of claim 9, wherein the soft tissue is wrinkled, scarred, depressed, cheek, or lip skin.

11. The method of claim 9, wherein the soft tissue is depressed due to a chronic disease, trauma, or surgical removal.

12. A method for repairing, reconstructing, or reinforcing a damaged or weakened tissue or organ in a subject, the method comprising:
providing a blood vessel patch sized to fit a damaged or weakened area in the tissue or organ;
attaching the blood vessel patch to the area; and
optionally attaching a vascularized soft tissue flap to the patch, thereby repairing, reconstructing, or reinforcing a damaged or weakened tissue or organ.

13. The method of claim 12, wherein providing the blood vessel patch comprises:
providing a blood vessel or portion thereof; and
slicing a portion of a blood vessel longitudinally to provide a patch.

14. The method of claim 12, wherein the damaged or weakened tissue or organ is a luminal structure of the aerodigestive tract such as the laryngeal, pharyngeal or esophageal passages.

15. The method of claim 12, wherein the damage is the result of trauma or a surgical procedure to remove a lesion.

16. The method of claim 10, wherein the lesion is a malignant or benign tumor.

17. The method of claim 7, wherein the defect is due to a congenital malformation.

18. The method of claim 12, wherein the damaged or weakened tissue or organ is a stenotic region of a laryngeal, pharyngeal or esophageal, passage, and the method comprises:
incising a stenotic region of at least one of a larynx, a pharynx, or an esophagus;
preparing a blood vessel patch sized to expand the stenotic region; and
affixing at least one blood vessel patch to the incised region to increase a luminal caliber of the larynx, pharynx or esophagus.

19. The method of claim 18, further comprising freezing and thawing the blood vessel patch prior to affixing the blood vessel patch to the incised region.

20. The method of claim 12, wherein the blood vessel or portion thereof is an aorta or portion thereof.

21. The method of claim 12, comprising attaching a vascularized soft tissue flap to the patch.

22. The method of claim 12, wherein at least a portion of the damaged or weakened area is vascularized soft tissue, and the patch is at least partially attached to the vascularized soft tissue.

23. The method of claim 2, wherein the blood vessel or portion thereof is cryopreserved.

24. The method of claim 12, wherein the blood vessel patch is cryopreserved.
